SEQUENCE LISTING (2 sequences)

SEQ ID NO: 1 - DNA, 2364 bp, Candida albicans
  CDS (1)..(2364)
  gene (1)..(2364): gene CaNL256
atgttgaaaaacgataccgttttcactaaagatatttcttgtacggcgataactggtaaagatgcctggaatcggccaacaccacaaccaatcactatatcattatctttcaatactgatttccataaggcatcggaattggataatttgaaatactcaattaattatgctgttattaccagaaatgtaactgaatttatgaaatcaaatgagcatttaaatttcaagtcattaggaaatattgctcaagcaattagtgatattggattagatcaatctagaggtggtggatctattgtggatgtgacgataaaaagtttgaaatcagaaataagagctgaaagtgtcgaatataaaattaatagaaacactttgggtcaacccgttccattagatattttccaagttaataaattgagattattgacgattattggagttttcacatttgaaagattacaaaaacaaatagttgatgttgatttgcaatttaaaattgaacctaattccaatttatatttccatcaaataattgctgatattgtttcatacgtggaatcatctaatttcaaaactgtagaagcattggtgtctaagattggtcaattgacatttcagaaatatgacggagtagctgaagttgttgctactgtcactaaaccgaatgcatttagtcatgttgaaggtgttggagtatcatctaccatggtcaaagacaatttcaaagatatggaaccagttaaatttgaaaacacaattgctcaaactaatagagcattcaatttacctgttgaaaatgagaaaactgaggattataccgggtaccacactgcatttattgcctttggatccaatactggaaatcaagtagaaaatattaccaattcattcgaattgttgcaaaaatatggaatcaccatagaagcaacttcatcattgtacatttctaaaccaatgtattacttggatcaaccagattttttcaatggagtaattaaagtgaatttccaaaacatttcacctttccagttgttgaaaattctaaaagatattgaatataaacatttagaaaggaaaaaagactttgataatgggcccagatcaatagatttggatattatactatatgacgatttacaattaaataccgagaatctaattattccacataaatcaatgttagaaagaacatttgtattacaaccattatgtgaagtattgccccctgattatattcatcccatcagtgcagaaagtttgcatagccatttacaacaattaataaatgataaacctcaagagacagtacaagaatcgtctgatttattacaatttatcccagtctctagattgcctgtcaaagataatattttgaaatttgatcaaattaatcataaatctcctactttgattatgggtatattgaatatgactcctgattcatttagtgatggtgggaaacattttggaaaagaactagataatactgtgaagcaggcagagaaattagtcagtgagggtgctacgattattgacattggaggagtttccacacgcccaggaagtgttgaacccactgaggaagaagaattggaacgtgtgattccattaattaaagctattcgtcaatcactgaaccctgatttactgaaggtgttgatttcggttgatacttatcgtaggaacgttgctgaacaaagtttacttgtgggtgctgacataatcaacgatatctcaatgggcaaatatgatgaaaaaatatttgatgtggttgctaaatacggatgtccttatatcatgaatcatactcgaggatcacctaaaaccatgtctaaattgaccaattatgaatcaaatacaaatgatgatattatcgaatatataattgatcctaaattaggacatcaagaattggatttgtcacctgaaatcaagaatttactcaatggaatcagtcgtgaattgagtttacaaatgtttaaagccatggctaaaggagtgaaaaaatggcaaattattttggatcctggtattggatttgctaaaaatttgaatcaaaatttagcagttattcgtaatgcctcgttttttaaaaaatattctattcaaattaatgaacgtgttgatgatgtgacaatcaaacataaatatttaagttttaatggtgcttgtgttttggtggggacatcaagaaagaagtttttggggacattaactggtaatgaagtgcctctggatcgagtatttggcactggtgcaacagtgtctgcgtgtattgaacaaaacactgatattgtaagagttcatgatgttaaagaaatgaaagatgtagtatgtataagtgatgcaatttataaaaatgta

SEQ ID NO: 2 - protein, 788 aa, Candida albicans
MLKNDTVFTKDISCTAITGKDAWNRPTPQPITISLSFNTDFHKASELDNLKYSINYAVITRNVTEFMKSNEHLNFKSLGNIAQAISDIGLDQSRGGGSIVDVTIKSLKSEIRAESVEYKINRNTLGQPVPLDIFQVNKLRLLTIIGVFTFERLQKQIVDVDLQFKIEPNSNLYFHQIIADIVSYVESSNFKTVEALVSKIGQLTFQKYDGVAEVVATVTKPNAFSHVEGVGVSSTMVKDNFKDMEPVKFENTIAQTNRAFNLPVENEKTEDYTGYHTAFIAFGSNTGNQVENITNSFELLQKYGITIEATSSLYISKPMYYLDQPDFFNGVIKVNFQNISPFQLLKILKDIEYKHLERKKDFDNGPRSIDLDIILYDDLQLNTENLIIPHKSMLERTFVLQPLCEVLPPDYIHPISAESLHSHLQQLINDKPQETVQESSDLLQFIPVSRLPVKDNILKFDQINHKSPTLIMGILNMTPDSFSDGGKHFGKELDNTVKQAEKLVSEGATIIDIGGVSTRPGSVEPTEEEELERVIPLIKAIRQSLNPDLLKVLISVDTYRRNVAEQSLLVGADIINDISMGKYDEKIFDVVAKYGCPYIMNHTRGSPKTMSKLTNYESNTNDDIIEYIIDPKLGHQELDLSPEIKNLLNGISRELSLQMFKAMAKGVKKWQIILDPGIGFAKNLNQNLAVIRNASFFKKYSIQINERVDDVTIKHKYLSFNGACVLVGTSRKKFLGTLTGNEVPLDRVFGTGATVSACIEQNTDIVRVHDVKEMKDVVCISDAIYKNV

(19) United States
(12) Patent Application Publication
(10) Pub. No.: US 2006/0088900 A1
(43) Pub. Date: Apr. 27, 2006

(54) Method for determining inhibition activity of a compound on one of the enzymes of the folate synthesis pathway

(76) Inventors: Therese Stachyra, Villers Sur Marne (FR); Jacques Biton, La Croix Saint Ouen (FR)

Correspondence Address: ROSS J. OEHLER;AVENTIS PHARMACEUTICALS INC., ROUTE 202-206, MAIL CODE: D303A, BRIDGEWATER, NJ, 08807, US

(21) Appl. No.: 10/485,346

(22) PCT Filed: Jul. 30, 2002
(86) PCT No.: PCT/EP02/09421

§ 371 Date: Jun. 8, 2004

(30) Foreign Application Priority Data
Aug. 1, 2001 (EP) 01402085.3

(51) Int. Cl. C12Q 1/42

(52) U.S. Cl. 435/21

(57) ABSTRACT

The invention is directed to a method for determining the inhibition activity of a compound to be tested on at least the DHPS activity of the folate synthesis pathway, which is coupled with the detection of the release of formed inorganic phosphate. An enzymatic assay, implementing this method of determination on an enzymatic composition, is also within the scope of the invention. This assay is suitable for high throughput screening of compounds affecting folate pathway.

### FIELD OF THE INVENTION

The invention is directed to a method for determining the inhibition activity of a compound to be tested on at least the DHPS activity of the folate synthesis pathway, which is coupled with the detection of the release of formed phosphate.

### BACKGROUND OF THE INVENTION

Reduced folate cofactors are essential for the synthesis of purines, thymidilate, glycine methionine, panthotenic acid and N-formyl methionyl-tRNA. Folates are vitamins for humans while most microbial cells must synthesize folates de novo since they lack the carrier mediated active transport system of mammalian cells that allows the use of preformed dietary folates.

In P.carinii (Volpe et al 1995), S.cerevisiae (Sen-Gupta et al, 1997), and C.albicans, one gene encodes for three of the enzymes of the folate de novo pathway. The fol 1 gene from C.albicans codes for a putative protein of 88.6 kDa (788 amino acids), see PCT Application WO00/15838 (CaNL 256), see SEQ ID 1.

The Fol 1 protein sequences show significant homology with proteins to be involved in the biosynthesis of dihydropteroate (Lopez & Lacks, 1993, Slock et al, 1990) in both eukaryotic and prokaryotic microorganisms.

The fol 1 gene from P.carinii, S.cerevisiae and C.albicans encodes a multifunctional enzyme that catalyses the last three consecutive steps of the 7,8-dihydropteroate biosynthesis pathway (see FIG. 1). The first activity is the 7,8-dihydroneopterin aldolase or DHNA which catalyses the conversion of 7,8-dihydroneopterin to 6-hydroxymethyl-7,8-dihydropterin. The second activity is the 7,8-dihydro-6-hydroxymethylpterin-pyrophosphokinase or HPPK; it converts the product of DHNA reaction to 6-hydroxymethyl-7,8-dihydropterin pyrophosphate. The third activity is the dihydropteroate synthase or DHPS, it catalyses the condensation of para-aminobenzoic acid (pABA) with the 6-hydroxymethyl-7,8-dihydropterin pyrophosphate to form 7,8-dihydropteroate.

With no mammalian counterparts, these enzymes are attractive chemotherapeutics targets, since it is possible to target highly selective drugs against them for treating microbial diseases. A number of compounds have been developed as antimicrobial agents which target two of the seven enzymes in the folate pathway. Trimethoprim inhibits dihydrofolate reductase, thereby preventing the formation of tetrahydrofolate (Huovinen et al, 1995). The sulphonamides exert their antimicrobial effect on a different biosynthetic enzyme, dihydropteroate synthase (Slock et al, 1990).

Amino acid sequence comparisons and functional studies reveal marked diversity in DHNA, HPPK and DHPS structure and organization in the different species (Slock et al, 1990 ; Lopez & Lacks, 1993). Depending on the source, these three activities are expressed as a mono-, bi- or tri-functional enzyme on the same polypeptide. Taking into account the difficulty of producing multifunctional enzymes, in the case of multifunctional enzymes, the approaches in the literature were to isolate and express the monofunctional domain sequences with an aim to study the enzymatic system (Volpe et al, 1995).

Previous studies with monofunctional protein require the coupling of DHNA activity with extrinsic purified HPPK and DHPS activities, or the coupling of HPPK with extrinsic purified DHPS (Lopez & Lacks, 1993). Moreover, it also involves synthesis of the HPPK or the DHPS substrates.

Assays described in the literature (Lopez & Lacks, 1993, Volpe et al, 1995) are not amenable to high throughput screening (H.T.S). They generally used radiolabelled substrate: para-amino benzoic acid ([³H]PABA or [¹⁴C]pABA), that involves a separation step of the substrate from the products before counting the radioactivity. Formed radioactive dihydropteroate is separated from substrates by paper chromatography and measured in a scintillation counter (Volpe et al, 1995). The radioactive dihydropteroate can be separated from unreacted [³H] pABA by the ether extraction method before measuring the radioactivity in the scintillation counter. These steps, solvent extraction or paper chromatography, are not adaptable to H.T.S.

### SUMMARY OF THE INVENTION

The invention is directed to a method for determining the inhibition activity of a compound to be tested on one of the enzymes of the folate synthesis pathway, comprising the steps of incubation of said compound with an enzymatic composition having at least the DHPS activity, and of detection of the release of phosphate.

In a preferred embodiment, the DHPS activity is coupled with pyrophosphatase.

According to one embodiment of the invention, the enzymatic composition has a DHPS activity, or DHPS and HPPK activities, or DHPS and HPPK and DHNA activities.

This method of determination involves also the use of an (the) enzyme(s) which is (are) encoded by SEQ ID No. 1 or a homolog thereof and functional fragments thereof as well as by the corresponding encoded protein of SEQ ID No. 1 or a functional polypeptidic fragment thereof.

According to another embodiment, the method of determination is performed on an enzymatic composition which is derived from fungi, bacteria or protozoa.

The method of determination comprises an incubation step of the compound to be tested with 7,8-dihydroneopterin, ATP, pABA, Fol1, pyrophosphatase and a detection step of the release of phosphate. This can be applied by the measurement of DHPS activity but also of the DHNA and/or HPPK activities.

This method of determination allows the in vitro synthesis of the HPPK substrate, DHPS substrate and product.

The preferred embodiment in the present invention is the detection of phosphate by colorimetry method. However, detection of phosphate can also be performed by fluorometry or by radioactive means.

The invention is also directed to a method of screening of compounds with potential inhibitory effect on enzymatic composition by implementing the method of determination presented above.

Finally, the invention concerns an enzymatic assay for in vitro testing compounds for their inhibitory effect on an enzymatic composition as disclosed earlier, comprising the substrate 7,8-dihydroneopterin, and the Fol1 enzyme, and the enzyme pyrophosphatase, and PABA and ATP for simultaneous or sequential use.

### BRIEF DESCRIPTION OF THE FIGURES

FIG. 1 Scheme of the three reactions catalysed by Fol1.

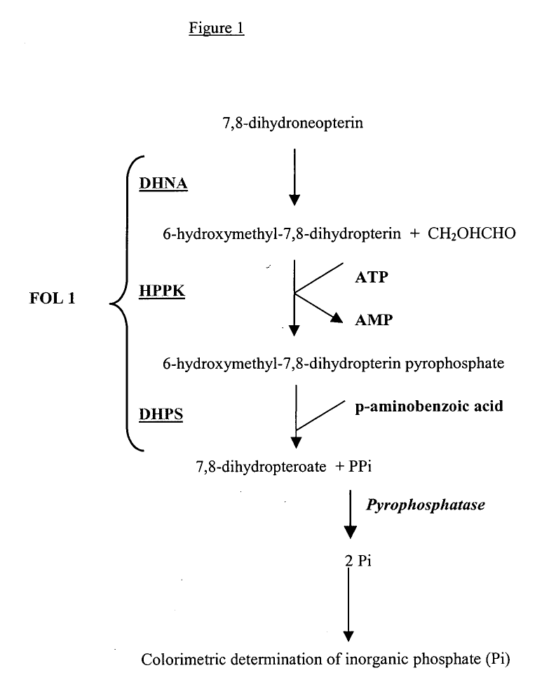

FIG. 2 Kinetics of DHNA+HPPK+DHPS, HPPK+DHPS and DHPS activities. The absorbance is measured at 850 nm; time scale is in minutes.

FIG. 3 In assay 1, kinetics of DHNA+HPPK+DHPS activities are measured at 850 nm in samples. After one hour incubation period, the substrate 7,8-dihydroneopterin is added to the samples with or without inhibitor 1.

FIG. 4 In assay 2, kinetics of HPPK+DHPS activities are measured at 850 nm in samples. After one hour incubation period, the substrate ATP is added to the samples with or without inhibitor 1.

FIG. 5 In assay 3, kinetics of DHPS activity are measured at 850 nm in samples. After one hour incubation period, the substrate PABA is added to the samples with or without inhibitor 1.

FIG. 6 Example of an inhibitor (inhibitor 2) that inhibits only the DHNA activity of Fol 1. Inhibition is observed only in condition 1 (assay 1/FIG. 6A). In conditions 2 and 3 (assay 2/FIG. 6B and 3/FIG. 6C respectively), doses of inhibitor 2 are added after accumulation of 6-hydroxymethyl-7,8-dihydropterin and 6-hydroxymethyl-7,8-dihydropterin pyrophosphate respectively, and before addition of ATP and p-aminobenzoic acid respectively.

### DETAILED DESCRIPTION OF THE INVENTION

The fol1 gene from C.albicans coding for DHNA, HPPK and DHPS, has been cloned and expressed in E.coli. The recombinant protein has been purified, and the enzymatic activity of this protein has been studied.

One object of the invention is to provide a simple and rapid method to screen a large number of compounds for their capability to inhibit specifically the Fol1 enzyme by the mean of inhibition of at least one of its 3 activities (DHNA, HPPK, and DHPS).

The invention is directed to a method and an enzymatic assay which involve a multifunctional protein Fol 1, which catalyses the sequential reactions by DHNA, HPPK, and DHPS. These robotics-based automated assay for measuring DHNA, HPPK, and DHPS coupled activities by inorganic phosphate detection are then adaptable to high throughput screen (H.T.S.) that can be used to discover new antibacterial, antifungal or antiprotozoa drugs.

These method and assay have been proved versatile as they allows to target either the 3 activities DHNA, HPPK and DHPS together (trifunctional protein) or only one of these activities (monofunctional proteins) . Consequently, the screening assay can be used to target antibacterial, antimicrobial or antiprotozoa enzymes.

The trifunctionality of Fol1 has been confirmed also by the HPLC and HPLC/MS methods.

Thus, this method for screening inhibitory compounds can be applied on enzymatic reactions which derive from the following organisms: bacteria, fungi or protozoa.

One preferred embodiment is directed to an enzymatic assay which is performed on recombinant Fol1 purified protein from C. albicans.

The measurement of DHNA, HPPK and DHPS activities is coupled with pyrophosphatase activity leading to the detection of inorganic phosphate. Colorimetric, fluorimetric or radioactive methods of inorganic phosphate detection can be used to detect inorganic phosphate. For example, it is possible to detect spectrophotometrically inorganic phosphate by the reaction catalyzed by uridine diphosphoglucose pyrophosporylase and coupling to phosphoglucose mutase and glucose R-phosphate dehydrogenase (Johnson, et al, 1968). According to one embodiment, a similar enzymatic reaction with uridine 5′-diphospho[¹⁴C]glucose can be used such as disclosed in Cheung & Suhadolnik, 1977.

According to another embodiment, the fluorometric method carries out the determination of inorganic phosphate by coupling the UDPG pyrophophorylase reaction with three other enzymes in a system of phosphorylation and double reduction to form NADPH as shown in Caines et al, 1984.

The preferred method for this screening assay is based on a calorimetric phosphate detection avoiding the use of radiolabelled substrates of DHNA, HPPK or DHPS. In order to accomplish this, the DHPS reaction is coupled with a pyrophosphatase to perform a detection.

As the use of a radioelement is optional, the Fol1 assay is adaptable to high throughput screening (H.T.S).

A spectrophotometric assay is used to measure the release of phosphate catalyzed by the pyrophosphatase which is added to the reaction mixture. The phosphate is detected by a calorimetric method based on formation of a phosphomolybdate complex (Upson et al, 1996 ; Baykov, 1989). This spectrophotometric assay is capable of automation.

The calorimetric reaction is monitored by measuring the increase in absorbance at 850 nm. The generation of two molecules of phosphate from each molecule of pyrophosphate increases the sensitivity of the assay, which has a linear range at least from 2 to 20 nmol of pyrophosphate in a reaction volume of 200 μl.

The assay of the present application is suitable for H.T.S. by following the three reactions catalysed by Fol1 as presented hereafter. The reaction medium contains the substrates: 7,8-dihydroneopterin, ATP and PABA. In the absence of inhibitor, the 3 enzymatic activities of Fol1 have different kinetics. The inventors have shown that at 37° C., the HPPK reaction is 2 times faster than the DHNA reaction and that the DHPS reaction is about 5 times faster than the HPPK reaction and 10 times faster than the DHNA reaction. Thus, in the absence of inhibitor, the 3 enzymatic activities of Fol1 have different kinetics.

This invention further provides method and reagents for in vitro synthesis of the HPPK substrate, the DHPS substrate and the DHPS product. As substrates of these enzymatic activities, only the 7,8-dihydroneopterin (DHNA substrate) is commercially available, the other substrates are unstable and difficult to prepare. This method thus avoids synthesizing chemically the 6-hydroxymethyl-7,8-dihydropterin (HPPK substrate) and the 6-hydroxymethyl-7,8-dihydropterin pyrophosphate (DHPS substrate).

The enzymatic compositions for the determination of enzymatic activities according to the present invention can be obtained from crude extracts or by genetic recombination. The preferred method of preparation is via genetic recombination. These techniques are well known for the skilled man.

Compounds are tested for a potential inhibitory effect by performing the assay in the three reaction conditions described hereafter. It is still within the scope of the invention to perform only 1 or 2 out of these 3 reactions, in particular when the enzymatic enzyme(s) affected by the inhibitor is determined.

The global inhibition assay compares activities of the three enzymes in three different reaction conditions. This global inhibition assay is performed in 3 different steps, referred below as assay 1, assay 2 and assay 3. Following these steps, if an inhibition is found with reactions conditions 1 but not with reactions conditions 2 and 3, the target of the inhibitor is the first enzymatic activity (i.e. DHNA) . If an inhibition is found with reaction conditions 2, but not 1 and 3, the inhibitor's target is the second enzymatic activity (i.e. HPPK) . If only the third assay shows inhibition with a potential inhibitor, the target is then the third activity (DHPS).

### EXAMPLES

Assay 1: Monitoring of the Combined Activities DHNA and HPPK and DHPS

The global reaction is followed monitoring the release of inorganic phosphate. The whole reaction is dependent on DHNA reaction which is the limiting reaction at 37° C.

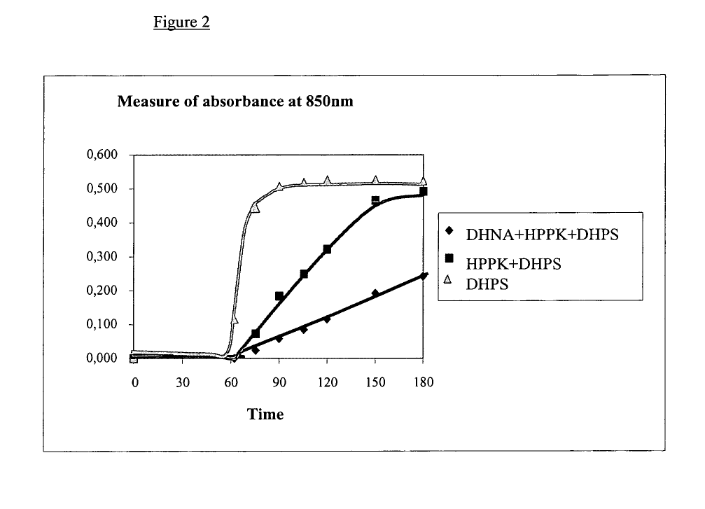

The kinetics of the Fol1 activities are given in FIG. 2.

During this assay (or screening 1) , the aim is to specifically find DHNA inhibitors (the slowest reaction). Total reaction medium and the putative inhibitor are used and the reaction is maintained at 37° C. for 120 minutes.

According to this assay, the 3 Fol1 activities (i.e. DHNA, HPPK and DHPS), are measured, by adding all substrates of enzymatic reactions (7,8-dihydroneopterin, ATP and pABA) to the reaction medium containing Fol1, pyrophosphatase and the inhibitor.

The final concentrations in the reaction medium are indicated in table 1.

TABLE 1

| | Final concentration in the | |
|---|---|---|
| | assay | |
| Tris/HCl buffer 44 mM pH 8.2 | 40 | mM |
| 2-mercaptoethanol 1 M | 10 | mM |
| MgCl₂ 0.5 M | 25 | mM |
| Pyrophosphatase 25 U/ml in casein 1 mg/ml | 2.5 | U/ml |
| ATP 10 mM | 100 | μM |
| p-aminobenzoic acid 2 mM | 75 | μM |
| Fol 1; 50 μg/ml in casein 1 mg/ml | 5 | μg/ml |
| DMSO 50% (+/− Inhibitor) | 5.00% | |
| 7,8-dihydro-D-neopterin 0.5 mM | 50 | μM |

NB: The final concentration in casein is 0.2 mg/ml

The reaction medium is: V_{f}=80 μl

Preparation of Enzymes Solution Stocks

Fol1 Solution 10×:

An aliquot of Fol1 at 9.6 mg/ml is diluted first at 200 μg/ml, and once more at 50 μg/ml, in Tris buffer containing casein 1 mg/ml.

The solutions must be prepared every day and stored in ice solution.

Solution 10× of Inorganic Pyrophosphatase

From a commercial flask of inorganic pyrophosphatase at 1000 U, a solution at 1000 U/ml is prepared in deionisated water (milliQ). Aliquots containing 20 μl at 1000 U/ml are stored at −80° C.

One aliquot is diluted at 25 U/ml in Tris buffer containing casein 1 mg/ml. The aliquot must not be conserved more than 10 h (one day).

The reaction medium is V_{f}=80 μl.

Pool 1 contains: Tris buffer; 2-mercaptoethanol, MgCl₂, ATP, p-aminobenzoic acid and the Fol 1 and pyrophosphatase enzymes prepared as described previously.

The reaction medium in the 96 well Microplate contains 64 μl pool 1, then is incubated for 60 min at 37° C. with shaking. This step is just to keep Fol1 in the same conditions as assay 2 and assay 3. Then, 8 μl inhibitor in DMSO 50% and 8 μl 7,8-dihydro-D-neopterin 0.5 mM are added to the reaction medium. The incubation of the reaction medium is carried out at 37° C. for 1 hour with shaking.

Stop Reaction

The Fol1 and pyrophosphatase reactions are stopped by acidification, when the reagent 1 used for detection of inorganic phosphate is added.

Blank (or Negative Control):

The reaction medium does not contain 7,8-dihydro-D-neopterin. It corresponds to the 0% activity control.

Positive Control:

The reaction medium does not contain inhibitor. It corresponds to 100% of activity.

“False Positives” Control:

This control is performed to monitor the inhibition of the inorganic pyrophosphatase or the calorimetric complex.

In this control, the 50 μM of 7,8-dihydroneopterin is replaced by 50 μM of inorganic pyrophosphate in the reaction medium containing the putative inhibitor.

Detection of Inorganic Phosphate (Molybdate-Arsenite Method): V_{f}=220 μl

The reagents for detection of inorganic phosphate (molybdate-Arsenite method) are the following:

Reagent 1 is composed of
6 volumes ammonium heptamolybdate 0.5% in H₂SO₄0.5 M
1 volume ascorbic acid 10%
2 volumes SDS 10%

Reagent 2 is composed of:
sodium citrate 2%
sodium meta-arsenite 2%
acetic acid 2%

Each well contains 80 μl reaction medium. A volume of 80 μl reagent 1 is added and the mixture is incubated at 37° C. for 5 min without shaking. Then, 60 μl reagent 2 is added to the mixture and the incubation is resumed at 37° C. for 10 min without shaking. The absorbance is read at 850 nm using a microplate detector (Molecular Device SpectraMax Plus).

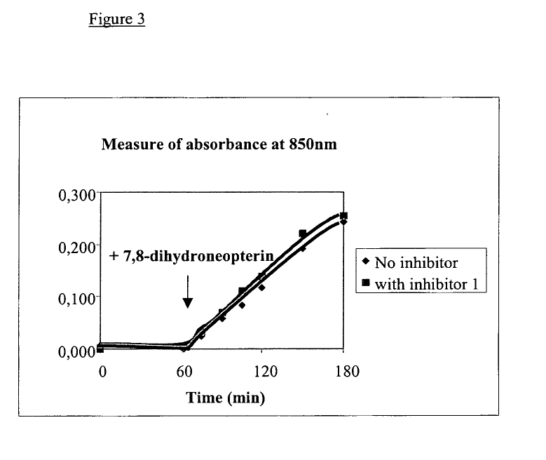

In view of the results shown in FIG. 3, there is no differences in terms of activities with or without the inhibitor 1, therefore no inhibition is detected on combined DHNA and HPPK and DHPS activities of Fol1.

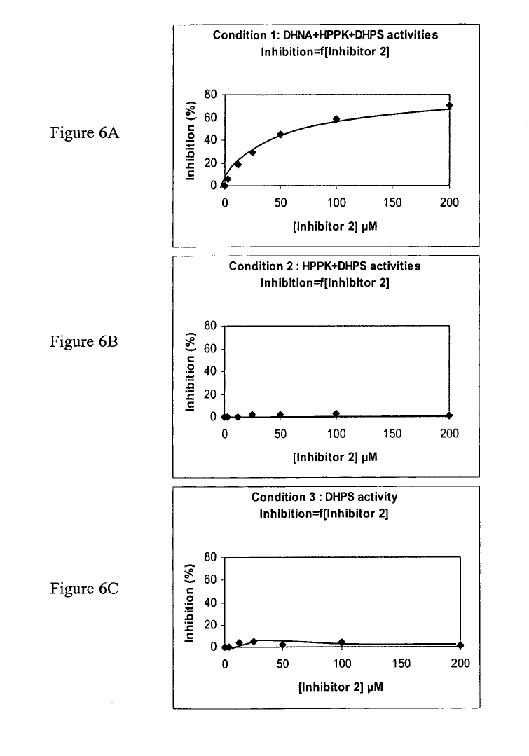

In direct contrast, the results of the assay 1 (condition 1) using inhibitor 2, demonstrates that inhibitor 2, inhibits one or more of the activities of Fol1 (DHNA or/and HPPK or/and DHPS (cf. FIG. 6)).

Assay 2: Monitoring of the Combined Activities HPPK and DHPS

Actually, the second assay measures the inhibition of activity of HPPK since this enzyme is the limiting reaction in terms of kinetics between HPPK and DHPS. In such conditions, (same substrates and enzymes as conditions used in assay 1, ATP omitted) the product of the first enzymatic reaction (6-hydroxymethyl-7,8-dihydropterin) is accumulated in the reaction medium for a defined period of time. The final concentrations in the reaction medium are identical to those used in assay 1.

The Reaction Medium is V_{f}=80 μl.

Pool 2 contains: Tris buffer; 2-mercaptoethanol, MgCl₂, 7,8-dihydro-D-neopterin, p-aminobenzoic acid and the 2 enzymes Fol 1, Pyrophosphatase prepared as described previously.

In the 96 well Microplate is delivered 64 μl pool 2. The medium is incubated for 60 min at 37° C. with shaking. During this step, the 7,8-dihydro-D-neopterin is totally converted in 6-hydroxymethyl-7,8-dihydropterin. Then, 8 μl inhibitor in DMSO 50% is added to the reaction followed by 8 μl ATP 1 mM. The reaction is carried out at 37° C. for 1 h with shaking.

The release of inorganic phosphate is monitored and the controls are performed as in assay 1.

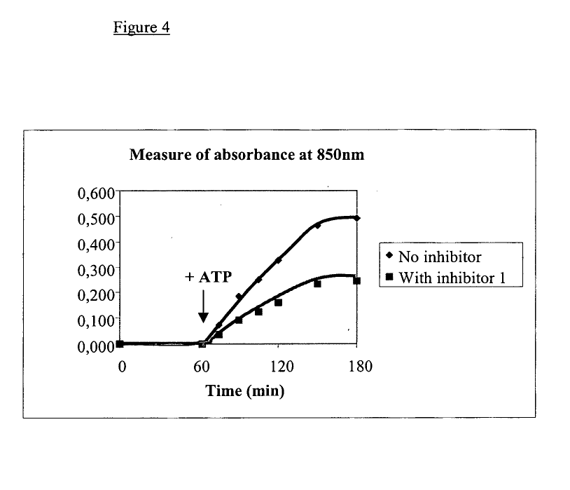

Results of the kinetics of combined HPPK and DHPS activities are shown in FIG. 4.

It appears clearly from these data that 50% of inhibition is detected on combined HPPK and DHPS activities of Fol1 when adding inhibitor 1.

In contrast, no inhibition is detected on combined HPPK and DHPS activities (condition 2) when adding inhibitor 2, showing that this inhibitor does not inhibit HPPK and/or DHPS activities (see FIG. 6).

Assay 3: Monitoring DHPS Activity

This assay measures the inhibition of activity of DHPS, using the same substrates and enzymes as in assay 1 without pABA.

The Reaction Medium is V_{f}=80 μl.

Pool 3 contains: Tris buffer; 2-mercaptoethanol, MgCl₂, 7,8-dihydro-D-neopterin, ATP, and the 2 enzymes Fol 1, Pyrophosphatase prepared as described previously.

In the 96 well Microplate is deliver 64 μl pool 3 and the incubation is carried out at 37° C. for 60 min with shaking. During this step, 7,8-dihydroneopterin is totally converted in 6-hydroxymethyl-7,8-dihydropterin pyrophosphate. A volume of 8 μl inhibitor in DMSO 50% is added to the reaction mixture, followed by 8 μl p-aminobenzoic acid 0.75 mM. The reaction is incubated at 37° C. for 1 h with shaking.

The detection of the inorganic phosphate release and the controls are performed such as in assay 1.

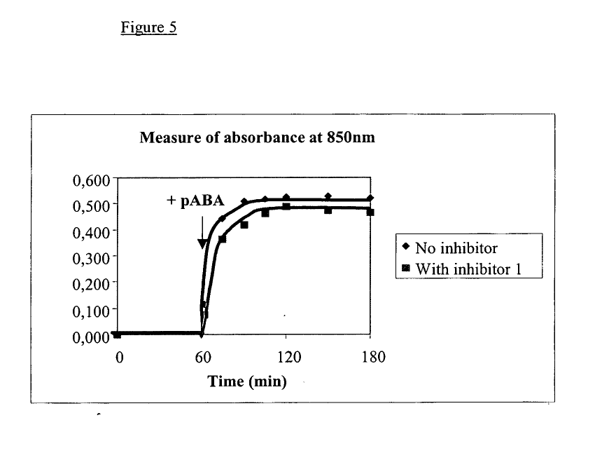

The results of kinetics of DHPS activity, with or without inhibitor 1, are presented in FIG. 5.

Data show that no inhibition is detected on the DHPS activity, so the inhibitor inhibits only the HPPK activity. It has been noticed that the HPPK inhibition cannot be detected in assay 1 because in this reaction condition, the DHNA reaction is the limiting step.

Concerning inhibitor 2, condition 3 data show that no inhibition is detected on the DHPS activity, so inhibitor 2 inhibits specifically the DHNA activity (see FIG. 6).

### References

Baykov A. Inorganic Pyrophosphate. Methods of enzymatic Analysis. Third edition, vol. VII: 558-566 (1989).

Caines P. S. M., Thibert R. J., and Draisey T. F. An improved fluorometric coupled enzymatic method for the determination of pyrophosphate in plasma and platelets: Microchemical Journal, 29, 168-181 (1984)

Cheung C. P., and Suhadolnik R. J. Analysis of inorganic pyrophosphate at the picomole level: Analytical biochemistry, 83, 61-63, (1977).

Johnson J. C., Shanoff M, Bass S. T., Boezi J. A. and Hansen P. G. An Enzymatic Method for Determination of Inorganic Pyrophosphate and Its Use as an Assay for RNA Polymerase: Anal. Biochem. 26, 137-145 (1968).

Huovinen P, Sundstrom L, Swedberg G, Skold O. Trimethoprim and sulfonamide resistance. Antimicrob Agents Chemother. Feb;39(2):279-89 (1995).

Lopez, P.; Lacks, S. A.: A bifunctional protein in the folate biosynthetic pathway of Streptococcus pneumoniae with dihydroneopterin aldolase and hydroxymethyldihydropterin pyrophosphokinase activities: J. Bacteriol., 175; 2214-2220 (1993)

Sen-Gupta M, Guldener U, Beinhauer J, Fiedler T, Hegemann J H. Sequence analysis of the 33 kb long region between ORC5 and SUIl from the left arm of chromosome XIV from Saccharomyces cerevisiae. Yeast. Jul;13(9):849-60 (1997).

Slock J, Stahly D P, Han C Y, Six E W, Crawford I P An apparent Bacillus subtilis folic acid biosynthetic operon containing pab, an amphibolic trpG gene, a third gene required for synthesis of para-aminobenzoic acid, and the dihydropteroate synthase gene. J Bacteriol;172,12, 7211-26 (1990).

Upson, R. H., Haugland, R. P., Malekzadeh, M. N.: A spectrophotometric method to measure enzymatic activity in reaction that generate inorganic pyrophosphate. Analytical Biochemistry, 243, 1, 41-5 (1996).

Volpe, F.; Ballantine, S. P.; Delves, C. J.: Two domains with amino-acid sequence similarity are required for dihydroneopterin aldolase function in the multifunctional folic acid synthesis Fas protein of Pneumocystis carnii: Gene, 160; 41-46 (1995)

1. A method for determining the inhibition activity of a compound to be tested on one of the enzymes of the folate synthesis pathway, comprising the steps of:
(i) incubating said compound with an enzymatic composition comprising a DHPS activity, and
(ii) detecting the release of phosphate.

2. The method of claim 1, wherein the enzymatic composition further comprises a pyrophosphatase, and step (ii) detects the release of inorganic phosphate.

3. The method of claim 1, wherein the enzymatic composition further comprises an HPPK activity or HPPK and DHNA activities.

4. The method of claim 3, wherein the DHPS activity is Fol1.

5. The method of claim 3, wherein at least one of the DHPS, HPPK or DHNA activities are encoded by
(i) SEQ ID No: 1 or a homolog or functional fragment thereof or
(ii) a nucleotide encoding the protein of SEQ ID No: 2 or a functional polypeptidic fragment thereof.

6. The method of claim 3, wherein the enzymatic composition is derived from fungi, bacteria or protozoa.

7. The method of claim 3, which comprises incubating the compound to be tested with 7,8-dihydroneopterin, ATP, pABA, Fol1, pyrophosphatase and detecting the release of inorganic phosphate.

8. The method of claim 3, wherein the enzymatic composition comprises a DHNA activity, and the DHNA activity is measured, the method comprising the incubation of the compound to be tested with 7,8-dihydroneopterin, ATP, pABA, Fol1, pyrophosphatase and the detection of the release of inorganic phosphate.

9. The method of claim 3, wherein the HPPK activity is measured in a single assay by:
(i) adding 7,8-dihydroneopterin and pABA , but with omission of ATP, to the enzymatic composition containing Fol1and pyrophosphate;
(ii) adding the compound to be tested and ATP when the product of the DHNA reaction, which is 6-hydroxymethyl-7,8-dihydropterin, is accumulated in the enzymatic composition after a defined period of time; and
(iii)monitoring the release of inorganic phosphate after the addition of the compound and ATP.

10. The method of claim 3, wherein the enzymatic composition comprises DHPS, HPPK and DHNA activities and the DHPS activity is measured in a single assay by:
(i) adding 7,8-dihydroneopterin and ATP but with omission of pABA, to the enzymatic composition containing Fol1and pyrophosphate;
(ii) adding the compound to be tested and pABA when the substrate of DHPS reaction, which is 6-hydroxymethyl-7,8-dihydropterin pyrophosphate, is accumulated in the enzymatic composition after a defined period of time; and
(iii) monitoring the release of inorganic phosphate after the addition of the compound and pABA.

11. The method of claim 1, wherein the enzymatic composition comprises a DHPS substrate is synthesized in vitro.

12. The method of claim 3, wherein the detection of phosphate is performed by colorimetry.

13. The method of claim 3, wherein the detection of phosphate is performed by fluorometry.

14. The method of claim 3, wherein the detection of phosphate is performed by radioactive labeling.

15. (canceled)

16. An Enzymatic assay kit for in vitro testing of compounds for their inhibitory effect on the folate synthesis pathway, the kit comprising:
(i) the substrate 7,8-dihydroneopterin, and
(ii) the Fol1 enzyme, and
(iii) the enzyme pyrophosphatase, and
(iv) pABA and ATP for simultaneous or sequential use.
